(19) United States
(12) Patent Application Publication
(10) Pub. No.: US 2023/0084621 A1
(43) Pub. Date: Mar. 16, 2023

(54) WIRELESS POWER TRANSFER FOR VENTRICULAR ASSIST DEVICE USING MAGNETICALLY COUPLED RESONATORS

(71) Applicant: Windmill Cardiovascular Systems, Inc., Austin, TX (US)

(72) Inventor: Erik Rist Larson, Austin, TX (US)

(21) Appl. No.: 17/929,289

(22) Filed: Sep. 1, 2022

Related U.S. Application Data
(60) Provisional application No. 63/240,239, filed on Sep. 2, 2021.

(51) Int. Cl. A61M 60/178; A61M 60/873; H02J 50/10; H02J 50/00

(52) U.S. Cl. CPC A61M 60/178; A61M 60/873; H02J 50/10; H02J 50/005; A61M 2205/8243

(57) ABSTRACT

Introduced here are systems for facilitating wireless power transfer to devices that are implanted in living bodies. The wireless power systems described herein utilize inductive coupling between a pair of resonators—namely, a first resonator located external to a living body and a second resonator located internal to the living body—for efficient wireless power transmission. Each resonator can include a conductive loop with at least one interruption in which discrete capacitors are situated. Moreover, each resonator may include a magnetic core that shapes the magnetic field created by the corresponding conductive loop.

### CROSS-REFERENCE TO RELATED APPLICATIONS

This application claims priority to US Provisional Application No. 63/240,239, titled “Wireless Power Transfer for Ventricular Assist Device Using Magnetically Coupled Resonators” and filed on Sep. 2, 2021, which is incorporated by reference herein in its entirety.

### TECHNICAL FIELD

Various embodiments concern magnetically coupled resonators for facilitating wireless power transfer to devices implanted in human bodies.

### BACKGROUND

A ventricular assist device (“VAD”)—also referred to as a “circulatory support device”—is an implantable electromechanical device that includes a pump which moves blood from the lower chambers of the heart to the rest of the human body. VADs tend to be implanted in individuals who have weakened hearts or who have experienced heart failure. Note that VADs differ from artificial cardiac pacemakers (or simply “pacemakers”) in that VADs physically pump blood, whereas pacemakers deliver electrical impulses to the heart to cause the chambers to contract.

A VAD can operate in parallel to the left ventricle, right ventricle, or both ventricles of the heart. VADs that support the left and right ventricles may be referred to as left ventricular assist devices (“LVADs”) and right ventricular assist devices (“RVADs”), respectively. Meanwhile, VADs that support both lower chambers may be referred to as biventricular assist devices (“BIVADs”).

The pumps used in VADs can be divided into several main categories, namely, (i) pulsatile pumps that mimic the natural pulsing action of the heart and (ii) continuous-flow pumps that do not mimic the natural pulsing action of the heart. Pulsatile VADs normally use positive displacement pumps that move the blood through mechanical action. Continuous-flow VADs normally use centrifugal pumps or axial flow pumps that have a central rotor with permanent magnets stored therein. Electric currents running through coils contained in a continuous-flow VAD will apply a force to these permanent magnets, which in turn causes the central rotor to spin. In a centrifugal pump, the central rotor is normally shaped to accelerate the blood circumferentially and thereby cause the blood to move toward the outer rim of the pump. Meanwhile, in an axial flow pump, the central rotor is normally cylindrical with blades that are helical, which causes the blood to be accelerated in the direction of axis of the central rotor.

Because VADs play a critical role in ensuring that blood is circulated to the body, it is important that power be reliably available. Historically, a cable—also referred to as a “percutaneous driveline” or simply “driveline”—has been interconnected between the VAD and a control unit that is external to the human body. The control unit is responsible for controlling power supplied by a battery—also external to the human body—in order to manage the VAD. However, the exit site through which the driveline leaves the human body is prone to infection. In fact, research has shown that driveline inventions are one of the issues that most frequently manifests following implantation of VADs.

### BRIEF DESCRIPTION OF THE DRAWINGS

FIG. 1 includes a high-level diagram of a wireless power system that is able to achieve wireless power transmission.

FIG. 2A includes top and side views of a first resonator (also referred to as an “external resonator”) that, in operation, is external to a living body.

FIG. 2B includes top and side views of a second resonator (also referred to as an “internal resonator”) that, in operation, is internal to the living body.

FIG. 3 illustrates how power transfer efficiency varies with frequency at different distances between external and internal loop resonators.

FIG. 4 illustrates how power transfer efficiency compares to distance between the external and internal loop resonators.

FIG. 5 illustrates how load power compares to load resistance.

FIG. 6A includes a perspective view of a compact resonator that includes conductive loops, a plurality of capacitors, a drum- or spool-shaped magnetic core, a coupling coil, and a substrate on which the conductive loops are arranged.

FIG. 6B includes a top view of the compact resonator of FIG. 6A.

FIG. 6C includes a side view of the compact resonator of FIG. 6A.

FIG. 6D includes a cross-sectional view of the compact resonator of FIG. 6A.

FIG. 7A includes a perspective view of a compact resonator that includes a conductive loop, a plurality of capacitors, a toroidal magnetic core, and a coupling coil.

FIG. 7B includes a top view of the compact resonator of FIG. 7A.

FIG. 7C includes a side view of the compact resonator of FIG. 7A.

FIG. 7D includes a cross-sectional view of the compact resonator of FIG. 7A.

FIG. 8 includes a side view of a durable housing (also referred to as an “enclosure”) in which an internal loop resonator may be situated along with a receiver coil.

FIG. 9A includes a perspective view of an enclosure in which an internal loop resonator may be situated along with a receiver coil.

FIG. 9B includes a top view of the enclosure of FIG. 9A.

FIG. 9C includes a side view of the enclosure of FIG. 9A.

FIG. 10 includes an image of a prototype of a wireless power system that includes magnetically coupled resonators.

Embodiments are illustrated by way of example and not limitation in the drawings. While the drawings depict various embodiments for the purpose of illustration, those skilled in the art will recognize that alternative embodiments may be employed without departing from the principles of the technology. Accordingly, while specific embodiments are shown in the drawings, the technology is amenable to various modifications.

### DETAILED DESCRIPTION

Normally, driveline infections can be successfully managed with antibiotics and local wound care. Managing driveline infections can be a hassle, however. For example, individuals in whom VADs have been placed may need to return to healthcare facilities (e.g., emergency rooms, hospitals, or clinics) regularly for treatment. Driveline infections can also lead to more serious complications. For example, driveline inventions can progress to pocket infection that may require immediate VAD exchange or heart transplantation. The term “pocket infection” may refer to a situation where infection has progressed into the cavity or “pocket” in the human body in which the VAD is situated.

For these reasons, several entities have begun developing “fully implanted” VADs that are powered wirelessly. These VADs must be routinely connected to a power source external to the human body to recharge an implanted battery, though no driveline is necessary. While these VADs show promise in providing individuals with greater physical autonomy, questions remain regarding whether power can be consistently and reliably transferred from a power source to a VAD under different circumstances. For example, efficient power transfer may depend on precise alignment being maintained between the VAD and power source, but precise alignment can be difficult to maintain, especially over longer periods of time (e.g., several minutes). As another example, efficient power transfer may require that the VAD and power source be located in close proximity (e.g., within one or two inches) but such closeness may be difficult—and in some instances, impossible—to achieve.

Introduced here are magnetically coupled resonators for facilitating wireless power transfer to devices that are implanted in living bodies. For the purpose of illustration, the magnetically coupled resonators are described in the context of VADs implanted in human bodies. However, those skilled in the art will recognize that the magnetically coupled resonators could be used to wirelessly transfer power to other types of implantable devices. Moreover, those skilled in the art will recognize that the magnetically coupled resonators could be used to wirelessly transfer power to devices implanted in animal bodies, for example, in addition to human bodies.

As further discussed below, the wireless power system described herein utilizes inductive coupling between a pair of resonators—namely, a first resonator (also referred to as a “transmitter resonator”) located external to a living body and a second resonator (also referred to as a “receiver resonator”) located internal to the living body—efficient wireless power transmission. Through inductive coupling, the pair of resonators are able to achieve high efficiency at large distances (e.g., in excess of several inches). This may hold true even when the pair of resonators are offset from one another. Said another way, relatively efficient power transfer may be achievable even if the pair of resonators are not precisely aligned or located in close proximity with each other. In contrast, conventional wireless power transfer technologies that use inductive coupling between a pair of coils require that those coils be closely aligned in close proximity in order to achieve acceptable efficiency as mentioned above.

### Terminology

Brief definitions of some terms used throughout the Detailed Description are provided below.

References in this description to “an embodiment” or “some embodiments” means that the feature being described is included in at least one embodiment of the technology. Occurrences of such phrases do not necessarily refer to the same embodiment, nor are they necessarily referring to alternative embodiments that are mutually exclusive of one another.

The terms “comprise,” “comprising,” and “comprised of” are to be construed in an inclusive sense rather than an exclusive or exhaustive sense (i.e., in the sense of “including but not limited to”). The term “based on” is also to be construed in an inclusive sense rather than an exclusive or exhaustive sense. Accordingly, the term “based on” is intended to mean “based at least in part on” unless otherwise noted.

The terms “connected,” “coupled,” and variants thereof are intended to include any connection or coupling between two or more elements, either direct or indirect. The connection or coupling can be physical, electrical, or a combination thereof. For example, elements may be electrically coupled to one another despite not sharing a physical connection.

### Overview of Wireless Power Transfer Through Electromagnetic Induction

In electrical engineering, two conductors are said to be “inductively coupled” or “magnetically coupled” when those conductors are configured such that a change in current through one conductor (also called the “first conductor” or “transmitter conductor”) induces a voltage across the other conductor (also called the “second conductor” or “receiver conductor”) through electromagnetic induction. Conventionally, the transmitter and receiver conductors have taken the form of coils. As the current through the transmitter conductor changes, the magnetic field around the transmitter conductor will also change. This variation in the magnetic field will induce a voltage in the receiver conductor through electromagnetic induction. Thus, an alternating current in the transmitter conductor can generate a magnetic field that induces a voltage in the receiver conductor, and the voltage can be used to power a device, for example.

Inductive power transfer efficiency can be determined based on the level of coupling between the magnetic fields of the transmitter and receiver conductors, as well as the electrical properties of the transmitter and receiver conductors. High power transfer efficiency typically requires that the coupling between the transmitter and receiver conductors be high as the electrical properties of the transmitter and receiver conductors are generally limited. Accordingly, establishing good coupling of the transmitter and receiver conductors has historically been critical to achieving efficient power transfer. Moreover, poor coupling due to misalignment or separation greater than approximately 0.5 inches reduces power transfer efficiency and leads to heating of the coils and surrounding tissue.

As mentioned above, another factor that impacts power transfer efficiency is alignment of the transmitter and receiver conductors. Alignment can be difficult to consistently achieve in the context of implantable devices since the transmitter conductor will be located external to the living body while the receiver conductor will be located internal to the living body. Further, while the general position and orientation of the receiver conductor may be known, the precise position and orientation of the receiver conductor may not be known, resulting in further difficulty in achieving alignment between the transmitter and receiver conductors.

As further discussed below, the magnetically coupled resonators described herein can be designed to exhibit beneficial electrical properties that allow for high power transfer efficiency with reduced field coupling. This design may also allow for satisfactory power transfer efficiency even when the magnetically coupled resonators are not aligned along a common axis. High or satisfactory power transfer efficiency is defined as an overall system efficiency greater than 30-90 percent with less than a 2° C. temperature increase in surrounding tissue.

### Overview of Highly Resonant Magnetic Coupling

Highly resonant magnetic coupling was first demonstrated at the Massachusetts Institute of Technology in 2005. This was the first instance in which a pair of magnetically coupled resonators were used to efficiently exchange energy at greater distances than can be achieved using inductive coupling between a pair of coils. Resonators store and oscillate energy between the magnetic and electric fields at a specific frequency. Resonators with low resistive losses can efficiency exchange energy with reduced magnetic field coupling.

At a high level, highly resonant magnetic coupling relies on four elements: (1) a transmitter coil, (2) a first resonator that is magnetically coupled to the transmitter coil, (3) a second resonator, and (4) a receiver coil that is magnetically coupled to the second resonator. Applying energy (e.g., in the form of alternating current) to the transmitter coil can result in excitation of the first resonator, which in turn can result in excitation of the second resonator that induces energy (e.g., in the form of voltage) in the receiver coil. To efficiently transfer energy from the transmitter coil to the receiver coil, the first and second resonators may need to be precisely tuned to the same resonant frequency.

The first and second resonators are able to oscillate stored energy between magnetic and electric fields. Each resonator may comprise (i) an inductive element, (ii) a capacitive element, and (iii) an effective resistance. Current flows through the inductive element or coil to generate a magnetic field and terminates on the capacitive element where energy is stored in the electric field, alternatively at the resonant frequency. Resistance is undesirable and largely dependent on the properties of the material(s) that form the inductor and capacitor. Additionally, the geometry of these structures can greatly determine the resistance due to non-uniform current distribution. Energy can be exchanged between the first and second resonators through magnetic field coupling. In order to achieve high efficiency, however, the first and second resonators must not only have a high qualify factor (Q) and a good coupling coefficient (k), but must also be tuned to the same resonant frequency (f₀) as mentioned above and shown below:

$$Q = \frac{1}{R} \sqrt{\frac{L}{C}} = \frac{2 π {f}_{0} L}{R} = \frac{{f}_{0}}{b {ω}_{3 dB}} , k = \frac{M}{\sqrt{{L}_{1} {L}_{2}}} , {f}_{0} = \frac{1}{2 π \sqrt{L C}} , \qquad Eq . 1$$

where R is the effective resistance, L is the inductance, C is the capacitance, f₀ is the resonant frequency, 107 ₃ dB is the 3 decibel (dB) bandwidth, M is the mutual inductance between the first and second resonators, and L₁ and L₂ are the inductances of the first and second resonators, respectively. The effective resistance (R) is the sum of any resistive losses, magnetic losses, and dielectric losses.

The frequency of operation for highly resonant magnetic coupling is typically 6.78±0.015 megahertz (“MHz”) or 13.56±0.007 MHz as those frequency bands are allocated for industrial, scientific, and medical (ISM) purposes. However, highly resonant magnetic coupling could operate in other portions of the electromagnetic spectrum, for example, the radio spectrum that ranges from 1 hertz to 3,000 gigahertz. Operating in these high-frequency ranges has several advantages over operating in low-frequency ranges. First, operating at a higher frequency tends to result in a higher quality factor. Second, operating at a higher frequency will ensure that conductive (e.g., metal) objects proximate to the first and second resonators will not experience significant heating from eddy currents. This is particularly advantageous in the case of implantable devices, as it is generally undesirable to heat physiological structures internal to a living body unnecessarily. Third, operating at a higher frequency will lessen the amount of capacitance that is required to effect wireless power transfer.

Resistance at these higher frequencies may be affected by the magnetic field distribution, commonly referred to as the “skin effect” or “proximity effect.” The skin effect may result in a negligible reduction in conductor resistance with increased conductor cross-sectional area, while proximity may result in an increase in resistance with additional coil turns that counteract increases in inductance. These high-frequency effects limit the quality factor that can be achieved by conventional resonator designs (e.g., in coil form).

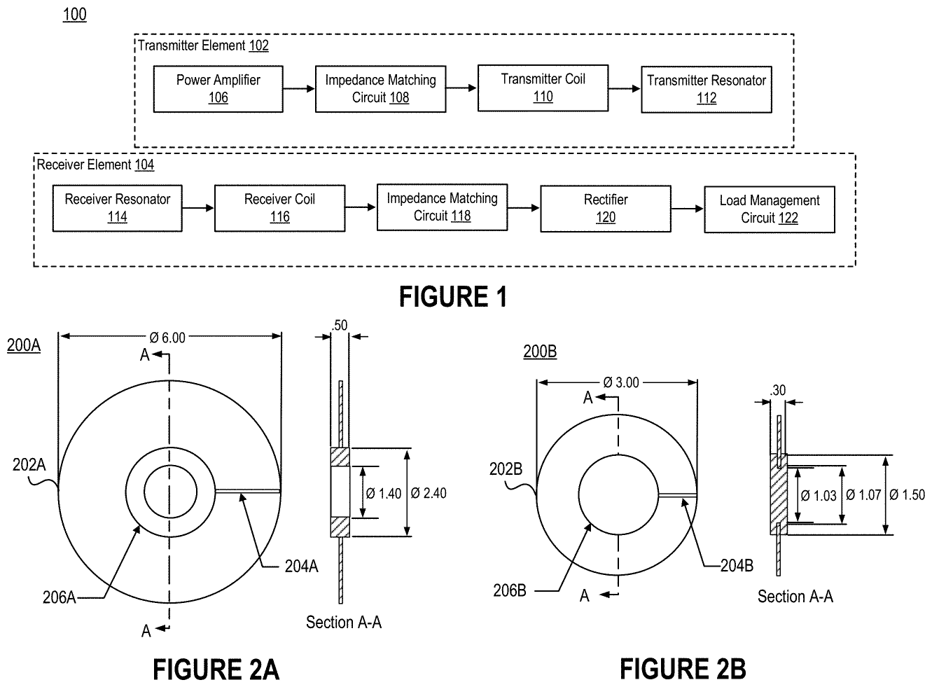

Note that a full system that is capable of transferring power will include other components in addition to those described above. FIG. 1 includes a high-level diagram of a wireless power system 100 that is able to achieve wireless power transmission. As shown in FIG. 1, the wireless power system 100 comprises a transmitter element 102 and a receiver element 104.

The transmitter element 102 can include a power amplifier 106, a first impedance matching circuit 108, a transmitter coil 110, and a transmitter resonator 112. The power amplifier 106 is an electrical circuit that is designed to drive a sinusoidal current at the resonant frequency of the transmitter resonator 112. The impedance matching circuit 108 may be responsible for implementing impedance matching, which is normally defined as the process of designing the input impedance or output impedance of a given electrical component (e.g., the transmitter element 102) to a desired value. Generally, the desired value is selected to maximize power transfer, though the desired value could be selected to minimize signal reflection. Here, impedance matching may be used to improve power transfer from the power amplifier 106 to the transmitter coil 110. At a high level, the transmitter coil 110 is simply representative of a coil through which power output by the impedance matching circuit 108 can flow. As further discussed below, the transmitter coil 110 can excite the transmitter resonator 112 by applying power—generally in the form of alternating current—thereto.

Meanwhile, the receiver element 104 includes a receiver resonator 114, a receiver coil 116, a second impedance matching circuit 118, a rectifier 120, and a load management circuit 122. At the receiver resonator 114, power may be induced through electromagnetic induction based on excitation of the transmitter resonator 112. The induced power—generally in the form of voltage—can be output by the receiver coil 116. As shown in FIG. 1, the induced power may be supplied to the second impedance matching circuit 118. The second impedance matching circuit 118 may be comparable to the first impedance matching circuit 108, except that the second impedance matching circuit 118 may be used to help power transfer from the receiver coil 116 to the rectifier 120. The rectifier 120 is an electrical component that is able to covert alternating current, which periodically reverses direction, to direct current, which flows in a single direction. This process—known as “rectification”—“straightens” the direction of current, and therefore can be used to generate direct current for use as a source of power. The load management circuit 122 may include one or more converters (e.g., boost converters or buck converters) that allow for optimization of power delivery. Specifically, the load management circuit 122 can be designed to optimize delivery of power output by the rectifier 120 to an implantable device (e.g., a VAD).

### Overview of Magnetically Coupled Resonators

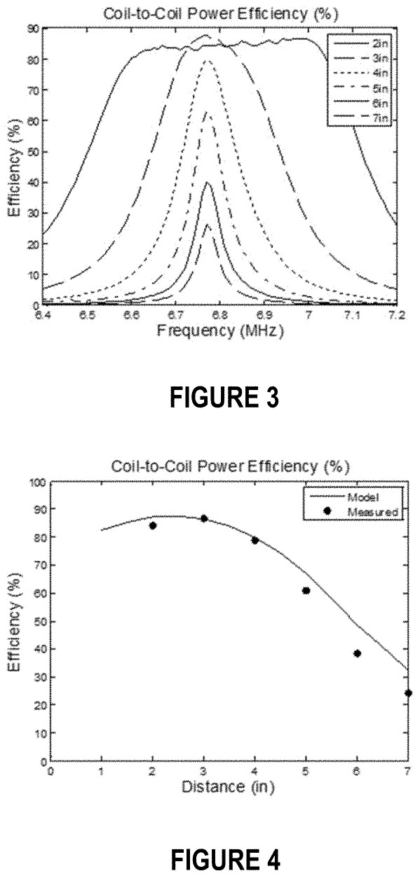

Introduced here are magnetically coupled resonators for facilitating wireless power transfer to devices implanted in human bodies. FIG. 2A includes top and side views of a first resonator 200A (also referred to as an “external resonator”) that, in operation, is external to a human body. FIG. 2B includes top and side views of a second resonator 200B (also referred to as an “internal resonator”) that, in operation, is internal to the human body. As mentioned above, the first and second resonators can operate in conjunction with one another such that power is wirelessly transferred from the first resonator to the second resonator via respective magnetic fields.

As shown in FIGS. 2A-B, each resonator may comprise a single conductive loop 202A-B with one or more interruptions 204A-B. Each interruption 204A-B may include discrete capacitors (e.g., in the form of a linear array) with high quality factors, as further discussed below. Capacitors can be soldered to both ends of each conductive loop to form a closed circuit. The conductive loops 202A-B may be substantially planar, so as to be in the form of annuluses with interruptions defined radially therethrough. As further described below, one or more capacitors may be situated within the interruption. In embodiments where the conductive loops 202A-B have non-negligible thickness (e.g., where the thickness is at least five percent of the diameter), the conductive loops 202A-B may be described as having the form of annular cylinders (also called “coaxial cylinders”) with interruptions defined radially therethrough.

The conductive loops 202A-B may be comprised of a highly conductive material, such as copper, silver, gold, or aluminum. The thickness of the conductive loops 202A-B may be greater than, or equal to, the skin depth of the conductive material at the resonant frequency at which the resonators 200A-B are to operate. For example, 1.5 ounce (oz) copper having a thickness of 2.06 mils may be used to form the conductive loops 202A-B, thus exceeding the skin depth of copper at 6.78 MHz which is 0.99 mils. The copper may be in the form of foil or sheets.

Moreover, each resonator may comprise a low-loss magnetic core 206A-B that is comprised of a ferromagnetic material that exhibits low magnetic losses, such as nickel-zinc (NiZn) ferrite. The conductive loops 202A-B and magnetic cores 206A-B may be designed to optimize wireless power transfer. For example, the radial dimensions of the conductive loops 202A-B and magnetic cores 206A-B may be optimized for power transfer efficient. Specifically, the conductive loop 202A included in the external resonator 200A may have a diameter between 3.0-10.0 inches (and preferably between 3.0-7.0 inches, such as 6.0 inches for example), and the conductive loop 202B included in the internal resonator 200B may have a diameter between 1.5-6.0 inches (and preferably between 2.0-4.0 inches, such as 3.0 inches for example). Meanwhile, the magnetic core 206A included in the external resonator 200A may have an outer diameter between 1.5-4.5 inches (and preferably between 2.0-3.0 inches, such as 2.4 inches for example) and an inner diameter between 0.5-2.5 inches (and preferably 1.0-2.0 inches, such as 1.4 inches for example), and the magnetic core 206B included in the internal resonator 200B may have an outer diameter between 1.0-4.0 inches (and preferably between 1.0-2.0 inches, such as 1.5 inches for example) and an inner diameter between 0.5-2.0 inches (and preferably 0.5-1.5 inches, such as 1.0 inches for example). Moreover, the gap between the inner diameter of the conductive loops 202A-B and outer diameter of the magnetic cores 206A-B may be relatively small (e.g., less than 0.05 inches, such as 0.01-0.02 inches) to increase the quality factor of the resonators 200A-B.

The magnetic cores 206A-B may extend axially away from the respective conductive loops 202A-B so as to shape the magnetic fields produced by the first and second resonators 200A-B. For example, the magnetic core 206A may extend axially away from a first plane that bisects the thickness of the corresponding conductive loop 202A, so as to shape the magnetic field produced by the external resonator 200A. Similarly, the magnetic core 206B may extend axially away from a second plane that bisects the thickness of the corresponding conductive loop 202B, so as to shape the magnetic field produced by the internal resonator 200B. Such a design is able to achieve a high quality factor by shaping the magnetic fields to reduce resistance near the inner radius of the conductive loops 202A-B, where density of the current is highest. The ferromagnetic material may also serve to increase inductance of the magnetic cores 206A-B. The combination of reduced resistance and increased inductance may result in a resonator with a high quality factor that also maintains a good coupling coefficient, making it suitable for wireless power transfer.

In some embodiments, the magnetic cores 206A-B are shaped to reduce the overall thickness of the resonators 200A-B. This may be particularly important for the internal resonator 200B as it will be contained in an implant. To decrease the thickness of the internal resonator 200B, a two-piece magnetic core may be employed that overlaps the inner diameter of the conductive loop 202B. For example, the magnetic core map overlap the inner diameter by 0.1-1.0 inches (and preferably 0.2-0.6 inches, such as 0.4 inches). The two-piece magnetic core shown in FIG. 2B could be used instead of the toroidally shaped magnetic core shown in FIG. 2A. Note while either design is possible for the magnetic cores 206A-B, the magnetic cores 206A-B generally have the same design for convenience (e.g., in manufacturing). The two-piece magnetic core has been shown to maintain power transfer efficiency over larger distances, while also reducing the thickness of the internal resonator 200B from roughly 0.7 inches to roughly 0.3 inches. This reduction in thickness is particularly advantageous for implantability. The external resonator 200A generally has a thickness of 0.2-1.2 inches (and preferably 0.2-0.6 inches, such as 0.5 inches), and the internal resonator 200B generally has a thickness of 0.2-1.2 inches (and preferably 0.2-0.4 inches, such as 0.3 inches). Note that the term “thickness” is generally interchangeable with the term “height” when used to describe dimensions of the external and internal resonators 200A-B or the magnetic cores 206A-B.

Additional changes to the shape and size of the magnetic core—in either the external resonator 200A or internal resonator 200B—may result in further improvements in performance (and reductions in dimensions). For example, the magnetic cores 206A-B may be shaped to optimize the coupling coefficient between the resonators 200A-B.

Fabrication of the first and second resonators 200A-B can be achieved using standard printed circuit board (PCB) processes, capacitors, and ferromagnetic materials. A copper-clad laminate material, such as a glass-reinforced epoxy laminate material (e.g., FR4), may be used to form the conductive loops. As an example, in embodiments where the conductive loops 202A-B are comprised of copper, the copper can be protected from oxidation via a solder mask or a plating comprised of gold and/or nickel. For instance, the conductive loops 202A-B may be coated with a double layer metallic coating comprised of nickel and gold through a process referred to as “electroless nickel immersion gold” or “ENIG.” As another example, the conductive loop 202A-B could be made on the sides of separate PCBs. Thus, the external resonator 200A may include a first PCB having an annular form on which the conductive loop 202A is made, and the internal resonator 200B may include a second PCT having an annular form on which the conductive loop 202B is made. Copper loops may be identical and with the interruption aligned such that capacitors are located on opposing sides. Capacitors from additional loops are electrically parallel to capacitors in other loops and sum to set the resonant frequency. In some embodiments only one side of the PCB includes a conductive loop, while in other embodiments both sides of the PCB include a conductive loop. Accordingly, two conductive loops could be made on opposing sides of a PCB. In such a scenario, the additional conductive loop may not increase the quality factor of the resonator but instead creates more space where additional capacitors can be placed.

Components of the first and second resonators 200A-B could also be specially designed, however. For example, the capacitors included in the interruptions 204A-B could be designed, selected, or arrayed to achieve a high quality factor. Advantageously, the capacitors should be able to handle induced currents or voltages while only adding minimal effective resistance to the formed circuit. Each capacitor will exhibit resistance, which is undesirable as it dissipates energy as heat and reduces the power transfer efficiency, and so selecting capacitors that only add minimal effective resistance is desirable. As another example, the magnetic cores 206A-B could be designed to optimally fit the central apertures of the conductive loops 202A-B, though the ferromagnetic material may be readily available. Several commercially available ferrites exhibit high permeability and low magnetic losses, making those ferrites an attractive option for the design shown in FIGS. 2A-B. Examples of ferrites that may be suitable include C2050, Ni—Zn ferrite produced by National Magnetics Group Inc., and 67, a Ni—Zn ferrite produced by Fair Rite.

Temperature stability of the resonators 200A-B is also important since a variation in temperature can cause a corresponding shift in resonant frequency, and thereby degrade efficiency of power transfer. Said another way, if the resonators 200A-B experience different variations in temperature, power transfer efficiency can degrade as the respective resonant frequencies may shift. The design shown in FIGS. 2A-B exhibits excellent temperature stability due to the properties of the capacitors—especially if the capacitors are ceramic “NP0” capacitors that have a temperature coefficient of zero and thus experience no drift—and ferromagnetic materials. Ferrite, for example, also has a low temperature coefficient.

Generally, embodiments are optimized to produce a high quality factor. However, that need not necessarily be the case. Higher power transfer efficiency may also be achievable by optimizing the geometry of the conductive loops 202A-B and magnetic cores 206A-B instead of, or in addition to, optimizing for overall efficiency of power transfer.

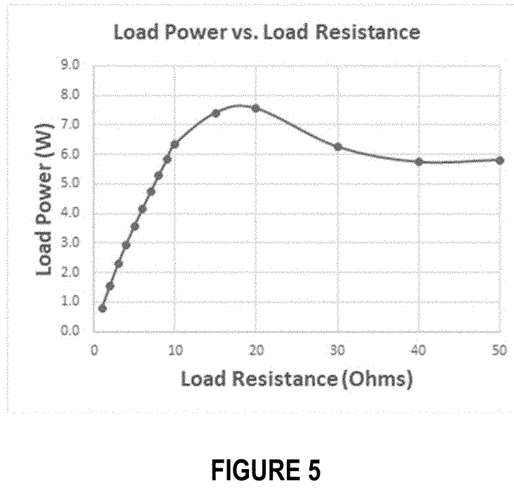

FIG. 3 illustrates how power transfer efficiency varies with frequency at different distances between external and internal “loop” resonators. Quasi-static finite element models may be used to model different embodiments by predicting values for inductance and resistance, based on the materials and geometry of the resonators. Finite element models are numerical approximates of a geometry that can estimate the inductance and resistance of the conductive loops. To set the resonant frequency for the magnetically coupled resonators, discrete capacitance values can be calculated, determined, or otherwise established based on the inductance values predicted by the quasi-static finite element models. Iterative assembly and testing by adding and removing capacitors may be necessary to tune the resonant frequency to the desired value.

As an example, assume that properties of external and internal loop resonators are predicted by quasi-static finite element models. Capacitance values can be selected to set the resonant frequency to a given value (e.g., 6.78 MHz). Measurements of resonant frequency, capacitance, and 3 dB bandwidth can then be used to derive the quality factor, inductance, and resistance as discussed above. Example values for an external loop resonator are provided in Table I, while example values for an internal loop resonator are provided in Table II. Quality factors are generally not well defined by manufacturers, however, and therefore can create some uncertainty that may provoke additional prototyping, testing, etc.

TABLE I
Example values for external loop resonator.

| Parameter | Model | Prototype |
|---|---|---|
| Capacitance (nF) | 4.7 | 5.24 |
| Inductance (nH) | 120 | 105 |
| Quality Factor | >800 | 850 |
| Resonant Frequency (MHz) | 6.780 | 6.779 |
| Resistance (Ohms) | 0.004 | 0.005 |

TABLE II
Example values for internal loop resonator.

| Parameter | Model | Prototype |
|---|---|---|
| Capacitance (nF) | 9.8 | 10.0 |
| Inductance (nH) | 57 | 55 |
| Quality Factor | >600 | 678 |
| Resonant Frequency (MHz) | 6.780 | 6.782 |
| Resistance (Ohms) | 0.003 | 0.0035 |

Various tests were performed using the external and internal loop resonators with the parameter values shown in Tables I and II.

FIG. 3 illustrates how power transfer efficiency compares to frequency when the external and internal loop resonators positioned in different arrangements. Here, there are six traces to indicate the power transfer efficiency when the external loop resonator is located two, three, four, five, six, and seven inches away from the internal loop resonator. At larger distances, the power transfer curve forms a single peak at roughly the resonant frequency. Accordingly, while power transfer may be most effective at smaller distances, power transfer efficiency may still be acceptable at larger distances (e.g., up to four or five inches).

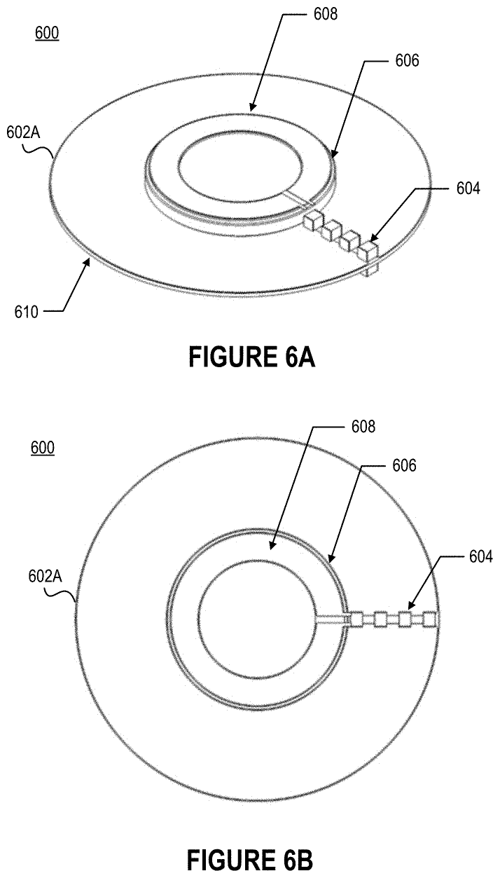

FIG. 4 illustrates how power transfer efficiency compares to distance between the external and internal loop resonators at a given frequency (e.g., 6.78 MHz). The power transfer efficiency generally decreases proportional to the distance. As can be seen in FIG. 4, there is generally good agreement between the model and measurements with RL=10 Ohms. RL is representative of the load resistance that is connected to the receiver coil coupled to the internal loop resonator. At a high level, the load resistance may be representative of the device to be powered via power output by the receiver coil.

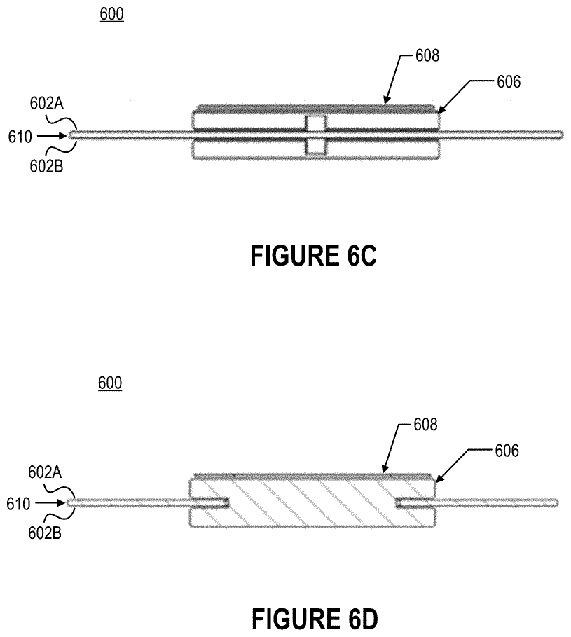

FIG. 5 illustrates how load power compares to load resistance. To generate the graph shown in FIG. 5, load power was measured with the external and internal loop resonators separated by three inches, as measured between the ends of the respective magnetic cores. The magnetically coupled resonators were then driven with a power amplifier. The receiving coil was connected to a rectifier, and the load resistance was varied. The load power measurements shown in FIG. 5 indicate that the magnetically coupled resonators can deliver sufficient power to not only operate an implanted device such as a VAD, but also recharge a battery connected thereto.

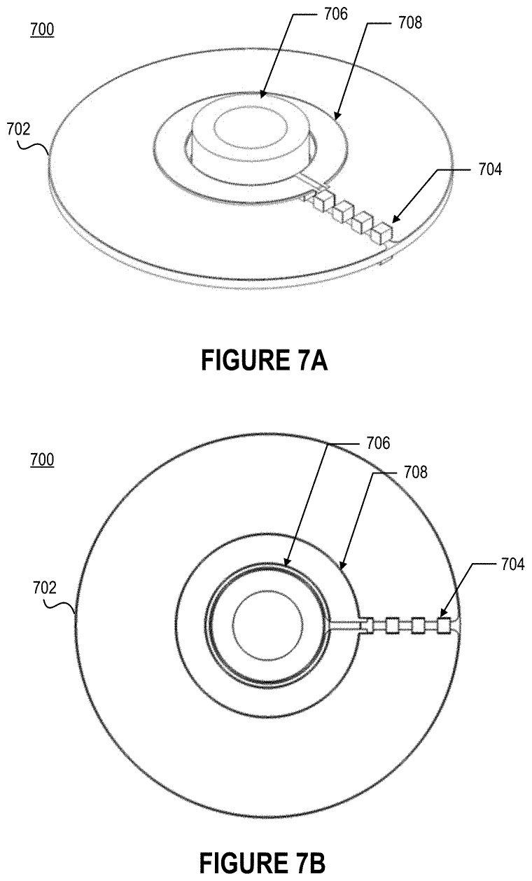

FIGS. 6A-C include perspective, top, and side views of a compact resonator 600 that includes conductive loops 602A-B, a plurality of capacitors 604, a drum- or spool-shaped magnetic core 606, a coupling coil 608, and a substrate 610 on which the conductive loops 602A-B are arranged. In this embodiment, the conductive loops 602A-B are layered on the opposing sides of the substrate 610. However, in other embodiments, a single conductive loop may be layered on one side of the substrate 610 as mentioned above. FIG. 6D includes a cross-sectional view of the compact resonator 600. Note that the design of the compact resonator 600 may be suitable for inclusion in a transmitter element (e.g., transmitter element 102 of FIG. 1) or a receiver element (e.g., receiver element 104 of FIG. 1).

Generally, the plurality of capacitors 604 are arranged in the form of a linear array as shown in FIGS. 6A-B. The capacitors 604 can be arranged within an interruption in each conductive loop 602A-B that extends radially away from the central aperture in which the magnetic core 606 is situated. The capacitors 604 could be ceramic “NPO” capacitors, for example. As discussed above, the number of capacitors 604—and their capacitance values—may depend on the desired quality factor or resonant frequency of the compact resonator 600.

In this embodiment, the magnetic core 606 is shaped like a drum or spool where the disc-like ends of the ferrite have a diameter that exceeds the inner diameter of the substrate 210 and conductive loops 602A-B. This ferrite shape allows for a more compact resonator while maintaining a high quality factor.

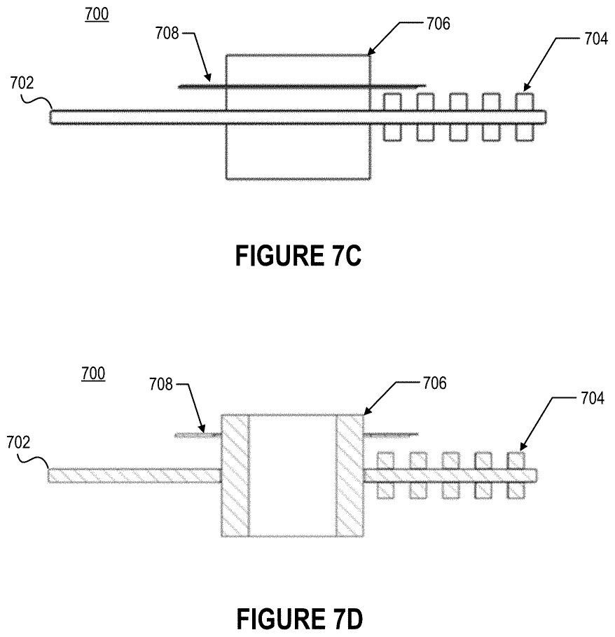

FIGS. 7A-C include perspective, top, and side views of a compact resonator 700 that includes a conductive loop 702, a plurality of capacitors 704, a toroidal magnetic core 706, and a coupling coil 708. FIG. 7D includes a cross-sectional view of the compact resonator 700. Again, the design of the compact resonator 700 may be suitable for inclusion in a transmitter element (e.g., transmitter element 102 of FIG. 1) or a receiver element (e.g., receiver element 104 of FIG. 1).

The compact resonator 700 of FIGS. 7A-D is largely comparable to the compact resonator 600 of FIGS. 6A-D. Here, however, the magnetic core 706 has a toroidal form. A solid disc may be used rather than a toroid without degrading performance. In geometry, a toroid is a surface of revolution with a hole defined through the middle. The axis of revolution passes through the hole and so does not intersect the surface. For example, when a rectangle is rotated around an axis parallel to one of its edges, then a hollow rectangle-section ring is produced as shown in FIG. 7D. The ring need not necessarily have a rectangular cross section, however. For example, the ring could have an ellipsoidal (e.g., circular) cross section, or the ring could have a rounded rectangular cross section where the edges are less sharp than those shown in FIG. 7D.

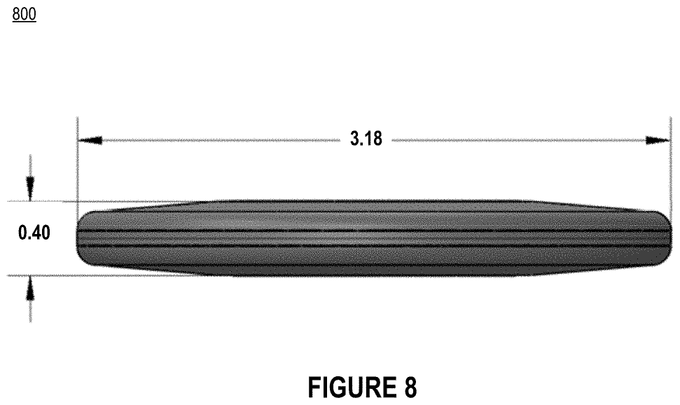

FIG. 8 includes a side view of a durable housing 800 (also referred to as an “enclosure”) in which an internal loop resonator (e.g., internal resonator 200B of FIG. 2B) may be situated along with a receiver coil. Generally, the enclosure 800 is comprised of a material that does not interact with the magnetic field of the internal loop resonator (or the magnetic field of the corresponding external loop resonator). For example, the enclosure 800 may be comprised of a ceramic material. The enclosure 800 could alternatively be comprised of plastic, though plastic enclosures may not be suitable for long-term implantability as it is difficult for plastic to be hermetic for extended periods of time. Enclosures for external loop resonators (e.g., external resonator 200A of FIG. 2A) can be comprised of a broader range of materials. For example, plastic can be used to construct enclosures for external loop resonators as those resonators are not implanted within living bodies.

Generally, the enclosure 800 is designed to fit within a relatively small cavity in the human body. For example, the enclosure 800 may have a diameter between 2.0-7.0 inches (and preferably 2.0-4.0 inches, such as 3.2 inches for example), and the enclosure 800 may have a thickness between 0.2-1.0 inches (and preferably 0.2-0.5 inches, such as 0.4 inches for example).

Together, the internal loop resonator and receiver coil may be designed to wirelessly receive power transferred from a source external to a living body and then provide the power to an implanted device (e.g., a VAD) as discussed above. The enclosure 800 could be situated in the same cavity as the implanted device, or the enclosure 800 could be situated in a different cavity than the implanted device.

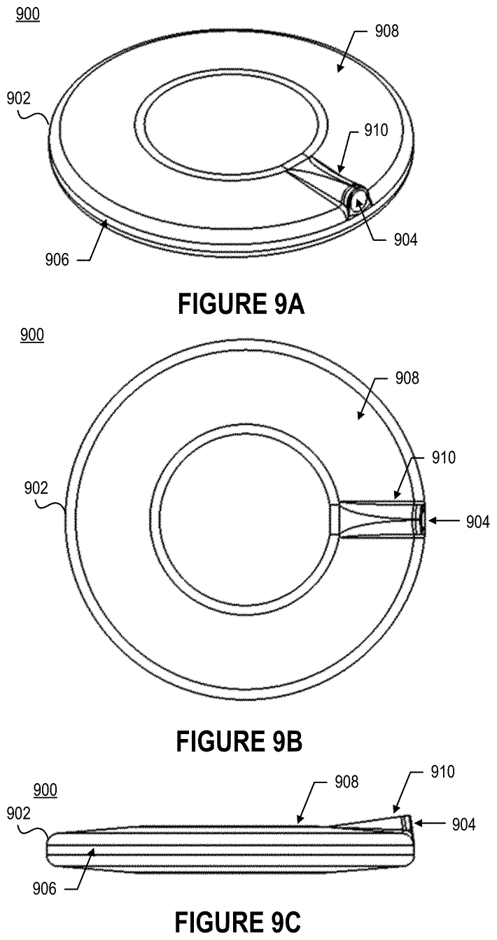

As further described with reference to FIGS. 9A-C, a wire may extend from a hermetic connector on the enclosure 800 to an implanted device (e.g., a VAD) to provide power. The enclosure 800 may be hermetically sealed so as to prevent fluids from entering the cavity defined therein. In addition to the internal loop resonator and receiving coil, a portion of the power conversion circuitry (e.g., impedance matching circuit 118, rectifier 120, and load management circuit 122 of FIG. 1) can be housed in the enclosure 800. Locating the appropriate power conversion circuitry inside the enclosure 800 may advantageously reduce resistive losses between the internal loop resonator and the next destination of the power.

FIGS. 9A-C include perspective, top, and side views of an enclosure 900 in which an internal loop resonator (e.g., internal resonator 200B of FIG. 2B) may be situated along with a receiver coil. As mentioned above, other components could also be situated inside the enclosure 900, such as an impedance matching circuit (e.g., impedance matching circuit 118 of FIG. 1), a rectifier (e.g., rectifier 120 of FIG. 1), a load management circuit (e.g., load management circuit 122 of FIG. 1), and other electronic components.

The enclosure 900 may have a structural body 902 that has a generally disc- or saucer-shaped form with rounded edges. This form is generally preferred as the “footprint” is relatively small when implanted in the living body. The enclosure 900 could have other forms, however. For example, the enclosure 900 could instead be in the form of a rectangular prism, pentagonal prism, hexagonal prism, ellipsoid, or hemisphere.

As shown in FIG. 9A-C, the enclosure 900 may have an aperture 904 through which a cable can extend. The cable may be interconnected between power conversion circuity located inside the enclosure 900 and the implanted device to which power is to be provided. In some embodiments, the aperture 904 is simply defined through the enclosure 900, for example, in the sidewall 906 so that the cable extends radially away from the center of the enclosure 900 or in the top cover 908 so that the cable extends axially away from the center of the enclosure 900. Alternatively, the enclosure 900 may be designed so that the cable does not extend from the center in a purely radial or purely axial manner. In FIGS. 9A-C, for example, a cable channel 910 (or simply “channel”) is defined along the top cover 908 of the enclosure 900. At a high level, the channel 910 serves as an entry for the cable, and therefore may also be referred to as a “cable entry.” Routing the cable through the channel 910 causes the cable to extend from the center of the enclosure 900 at an angle (e.g., of roughly 30 degrees). The angle of the channel 910 may be based on the intended or expected spatial relationship between the enclosure 900 and the implanted device to which power is to be provided.

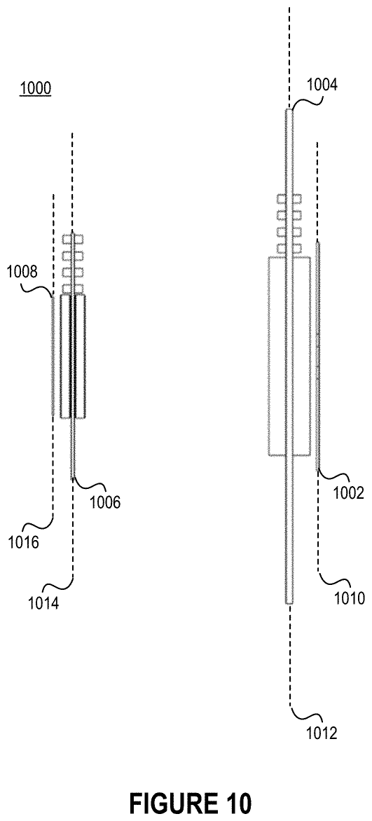

FIG. 10 includes an image of a prototype of a wireless power system 1000 that includes magnetically coupled resonators. The wireless power system 1000 may comprise a transmitter coil 1002 (also referred to as an “excitation coil”) and an external loop resonator 1004 that could be situated in a first durable housing that is external to a human body. As shown in FIG. 10, the transmitter coil 1002 may be situated, in the first durable housing, in a substantially parallel arrangement, such that a first plane 1010 that bisects the thickness of the transmitter coil 1002 is substantially parallel to a second plane 1012 that bisects the thickness of the external loop resonator 1004. The wireless power system 1000 may also comprise an internal loop resonator 1006 and a receiver coil 1008 that could be situated in a second durable housing that is internal to the human body. As shown in FIG. 10, the receiver coil 1008 may be situated, in the second durable housing, in a substantially parallel arrangement, such that a third plane 1014 that bisects the thickness of the internal loop resonator 1006 is substantially parallel to a fourth plane 1016 that bisects the thickness of the receiver coil 1008.

Power can be transferred from the first durable housing to the second durable housing—and, more specifically, from the external loop resonator 1004 to the internal loop resonator 1006—in accordance with the teachings herein. As mentioned above, power transfer efficiency may be highest when the external loop resonator 1004 and the internal loop resonator 1006 are aligned with one another in close proximity. Specifically, power transfer efficiency may be highest (i) when the first and second planes 1010, 1012 are substantially parallel to the third and fourth planes 1014, 1016 and (ii) the external loop resonator 1004 is proximate to the internal loop resonator 1006 (e.g., located within several inches). In accordance with the teachings herein, however, power transfer efficiency may still be acceptable when one—or both—of these conditions are not true. Accordingly, the wireless power system 1000 is able to better tolerate issues, such as misalignment and separation of the external loop resonator 1004 and the internal loop resonator 1006, that commonly occur.

In operation, the transmitter coil 1002 can excite the external loop resonator 1004 by applying a variable current thereto. The transmitter coil 1002 may be magnetically coupled to the external loop resonator 1004, which in turn may be magnetically coupled to the internal loop resonator 1006. As the current applied to the external loop resonator 1004 varies, the magnetic field of the external loop resonator 1004 will also vary. Because the external loop resonator 1004 is magnetically coupled to the internal loop resonator 1006 via the respective magnetic fields, varying the magnetic field of the external loop resonator 1004 will induce a voltage at the internal loop resonator 1006. The receiver coil 1008 can collect the voltage that is induced at the internal loop resonator and then provide the voltage to power conversion circuitry for conversion into a form suitable to power the implantable device. Specifically, the power conversion circuitry may receive, as input, the voltage that is induced at the internal loop resonator 1006 via the receiver coil 1008 and then produce, as output, a power signal in a form suitable for the implanted device. As discussed above with reference to FIGS. 9A-C, the power signal can be conveyed to the implanted device via a cable that extends through the enclosure in which the internal loop resonator 1006 and the receiver coil 1008 are housed. The power conversion circuitry could include, for example, the impedance matching circuit 118, rectifier 120, or load management circuit 122 of FIG. 1. Like the transmitter coil 1002 and external loop resonator 1004, the receiver coil 1008 may be magnetically coupled to the internal loop resonator 1006.

Note that the components of the wireless power system 1000 are shown in a particular arrangement for the purpose of illustration. These components can be redesigned and rearranged to be more suitable for implanting within a human body. As an example, the receiver coil 1008 could be miniaturized so that the second durable housing can more easily be implanted inside the living body.

### Remarks

The foregoing description of various embodiments of the claimed subject matter has been provided for the purposes of illustration and description. It is not intended to be exhaustive or to limit the claimed subject matter to the precise forms disclosed. Many modifications and variations will be apparent to one skilled in the art. Embodiments were chosen and described in order to best describe the principles of the invention and its practical applications, thereby enabling those skilled in the relevant art to understand the claimed subject matter, the various embodiments, and the various modifications that are suited to the particular uses contemplated.

Although the Detailed Description describes certain embodiments and the best mode contemplated, the technology can be practiced in many ways no matter how detailed the Detailed Description appears. Embodiments may vary considerably in their implementation details, while still being encompassed by the specification. Particular terminology used when describing certain features or aspects of various embodiments should not be taken to imply that the terminology is being redefined herein to be restricted to any specific characteristics, features, or aspects of the technology with which that terminology is associated. In general, the terms used in the following claims should not be construed to limit the technology to the specific embodiments disclosed in the specification, unless those terms are explicitly defined herein. Accordingly, the actual scope of the technology encompasses not only the disclosed embodiments, but also all equivalent ways of practicing or implementing the embodiments.

The language used in the specification has been principally selected for readability and instructional purposes. It may not have been selected to delineate or circumscribe the subject matter. It is therefore intended that the scope of the technology be limited not by this Detailed Description, but rather by any claims that issue on an application based hereon. Accordingly, the disclosure of various embodiments is intended to be illustrative, but not limiting, of the scope of the technology as set forth in the following claims.

What is claimed is:

1. A system for wirelessly transferring power, the system comprising:
a first resonator that, in operation, is external to a living body, the first resonator including—
a first conductive loop in the form of an annulus with an interruption defined radially therethrough,
a first plurality of capacitors that are situated in the interruption, and
a first magnetic core that is situated in the first conductive loop; and
a second resonator that, in operation, is internal to a living body, the second resonator including—
a second conductive loop in the form of an annulus with an interruption defined radially therethrough,
a second plurality of capacitors that are situated in the interruption, and
a second magnetic core that is situated in the second conductive loop.

2. The system of claim 1,
wherein the first resonator further includes a first circuit board in the form of an annulus with opposing sides,
wherein the first conductive loop is one of a first pair of conductive loops that are arranged along the opposing sides of the first circuit board,
wherein the second resonator further includes a second circuit board in the form of an annulus with opposing sides, and
wherein the second conductive loop is one of a second pair of conductive loops that are arranged along the opposing sides of the second circuit board.

3. The system of claim 1, further comprising:
a first coil that is configured to excite the first resonator through an application of current, the first coil being magnetically coupled to the first resonator; and
a second coil that is configured to receive energy from the second resonator by excitation of the first resonator, the second coil being magnetically coupled to the second resonator.

4. The system of claim 3, further comprising:
an enclosure in which the second coil and the second resonator are housed in a substantially parallel arrangement.

5. The system of claim 4, wherein the enclosure is hermetically sealed so as to prevent fluids from entering a cavity defined therein.

6. The system of claim 4, further comprising:
power conversion circuitry that is configured to receive, as input, the voltage from the second coil and produce, as output, a power signal in a form suitable for a device implanted in the living body; and
a cable that is electrically coupled to the power conversion circuitry,
wherein the cable extends through an aperture in the enclosure for coupling to the device to which the power signal is supplied.

7. The system of claim 1,
wherein first resonator further includes a first circuit board having an annular form on which the first conductive loop is situated, and
wherein the second resonator further includes a second circuit board having an annular form on which the second conductive loop is situated.

8. The system of claim 1, wherein the first and second magnetic cores are comprised of a low loss ferromagnetic material.

9. The system of claim 1,
wherein the first magnetic core extends axially away from a first plane that bisects a thickness of the first conductive loop, so as to shape the magnetic field produced by the first resonator, and
wherein the second magnetic core extends axially away from a second plane that bisects a thickness of the second conductive loop, so as to shape the magnetic field produced by the second resonator.

10. A receiver element for a wireless power system, the receiver element comprising:
a resonator that includes—
a conductive loop in the form of an annulus with an interruption defined radially therethrough,
at least one capacitor that is situated in the interruption, and
a magnetic core that shapes a magnetic field produced by the conductive loop; and
a coil that is configured to output a voltage induced at the resonator by excitation of another resonator included in a transmitter element that, in operation, is separated from the receiver element with a gap therebetween.

11. The receiver element of claim 10, wherein a plurality of capacitors are situated in the interruption in the form of a linear array.

12. The receiver element of claim 10, wherein capacitance values of the plurality of capacitors dictate a resonant frequency of the resonator.

13. The receiver element of claim 12, wherein the plurality of capacitors are selected and arrayed to handle the voltage while adding minimal effective resistance.

14. The receiver element of claim 10, wherein the conductive loop is comprised of copper.

15. The receiver element of claim 10, wherein the magnetic core is comprised of nickel-zinc ferrite.

16. The receiver element of claim 10,
wherein the conductive loop is comprised of a conductive material, and
wherein a thickness of the conductive loop is greater than, or equal to, a skin depth of the conductive material at a resonant frequency at which the resonator is to operate.

17. The receiver element of claim 10, wherein a gap between an inner diameter of the conductive loop and an outer diameter of the magnetic core is less than 0.05 inches.

18. The receiver element of claim 10, wherein an outer diameter of the magnetic core overlaps an inner diameter of the conductive loop.

19. The receiver element of claim 10, wherein a height of the resonator is 0.2-0.6 inches.

20. The receiver element of claim 10, further comprising:
power conversion circuitry that is configured to receive, as input, the voltage from the coil and produce, as output, a power signal; and
a cable that is electrically coupled to the power conversion circuitry; and
an enclosure in which the resonator, the coil, and the power conversion circuitry are housed,
wherein the enclosure includes an aperture through which the cable extends for coupling to a device to which the power signal is provided.

21. A resonator for facilitating wireless power transfer, the resonator comprising:
a conductive loop in the form of an annulus with an interruption defined radially therethrough;
at least one capacitor that is situated in the interruption; and
a magnetic core that shapes a magnetic field produced by the conductive loop.
